(19) United States
(12) Patent Application Publication
(10) Pub. No.: US 2021/0162106 A1
(43) Pub. Date: Jun. 3, 2021

(54) NEGATIVE-PRESSURE THERAPY UNIT WITH NOISE ATTENUATION AND INTEGRATED SEAL

(71) Applicant: KCI Licensing, Inc., San Antonio, TX (US)

(72) Inventors: James Killingworth SEDDON, Wimborne (GB); Benjamin Andrew PRATT, Poole (GB)

(21) Appl. No.: 17/254,586

(22) PCT Filed: Jun. 20, 2019
(86) PCT No.: PCT/US2019/038233

§ 371 Date: Dec. 21, 2020

Related U.S. Application Data
(60) Provisional application No. 62/691,431, filed on Jun. 28, 2018.

(51) Int. Cl. A61M 1/00

(52) U.S. Cl. CPC A61M 1/0088; A61M 2205/42; A61M 2205/8206; A61M 1/0066

(57) ABSTRACT

In some examples, a therapy unit for treating a tissue site may include an enclosure, a multi-function mount, and a pump. The enclosure may include at least one pneumatic conduit. The multi-function mount may be coupled within the enclosure and may include at least one pneumatic seal integrally formed as part of the multi-function mount and configured to provide a pneumatic seal relative to the at least one pneumatic conduit. The pump may be carried by the multi-function mount. Also provided are other apparatuses, systems, and methods suitable for treating a tissue site.

### RELATED APPLICATION

The present invention claims the benefit, under 35 U.S.C. § 119(e), of the filing of U.S. Provisional Patent Application Ser. No. 62/691,431, filed Jun. 28, 2018. This provisional application is incorporated herein by reference for all purposes.

### TECHNICAL FIELD

The invention set forth in the appended claims relates generally to tissue treatment systems that may be suitable for treating a tissue site with reduced pressure, and more particularly, but without limitation, to systems and apparatuses having improved noise attenuation, pneumatic sealing, and assembly configurations.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of a wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” and “vacuum-assisted closure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, the cost and complexity of negative-pressure therapy can be a limiting factor in its application, and the development and operation of negative-pressure systems, components, and processes continues to present significant challenges to manufacturers, healthcare providers, and patients.

### SUMMARY

New and useful systems and apparatuses related to tissue treatment are set forth in the appended claims. Illustrative embodiments are also provided to enable a person skilled in the art to make and use the claimed subject matter.

In some example embodiments, a therapy unit for treating a tissue site may include an enclosure, a multi-function mount, a power source, a pressure source, and a pneumatic coupler. The multi-function mount may be coupled within the enclosure. The power source and the pressure source may be carried by the multi-function mount. The pneumatic coupler may be configured to fluidly couple the pressure source to the therapy unit and may include an inlet expansion chamber within an inlet pathway and an outlet expansion chamber within an outlet pathway disposed through the pneumatic coupler.

Further, in some example embodiments, a therapy unit for treating a tissue site may include an enclosure, a multi-function mount, and a pump. The enclosure may include at least one pneumatic conduit. The multi-function mount may be coupled within the enclosure and may include at least one pneumatic seal integrally formed as part of the multi-function mount and configured to provide a pneumatic seal relative to the at least one pneumatic conduit. The pump may be carried by the multi-function mount.

Further, in some example embodiments, a therapy unit for treating a tissue site may include an enclosure, a pump, and a multi-function mount. The pump may be positioned within the enclosure. The multi-function mount may be coupled within the enclosure and may include a mount base and one or more capturing members extending outward from the mount base. At least one of the capturing members may be configured to contact the pump at a first contact surface and a second contact surface positioned non-coplanar to the first contact surface.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system that can provide negative-pressure therapy in accordance with this specification;

FIG. 2 is a perspective view of an example embodiment of a therapy unit that may be associated with some example embodiments the therapy system of FIG. 1;

FIG. 3 is an exploded, perspective view of the example therapy unit of FIG. 2;

FIG. 4 is a perspective view of an internal portion of the example therapy unit of FIG. 2, illustrating example embodiments of a multi-function mount and a pneumatic coupler in a partially assembled configuration within the therapy unit;

FIG. 5 is a perspective view of an internal portion of the example therapy unit of FIG. 2, shown in a fully assembled configuration;

FIG. 6 is a detail view of an example embodiment of pneumatic coupler that may be associated with some example embodiments of the therapy unit of FIG. 2;

FIG. 7 is a side view of the multi-function mount and the pneumatic coupler of FIG. 4, illustrating additional features that may be associated with some example embodiments.

### DESCRIPTION OF EXAMPLE EMBODIMENTS

The following description of example embodiments enables a person skilled in the art to make and use the subject matter set forth in the appended claims. Certain details already known in the art may be omitted. Therefore, the following detailed description is illustrative and non-limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as recognized by those skilled in the art, this frame of reference is merely a descriptive expedient rather than a strict prescription.

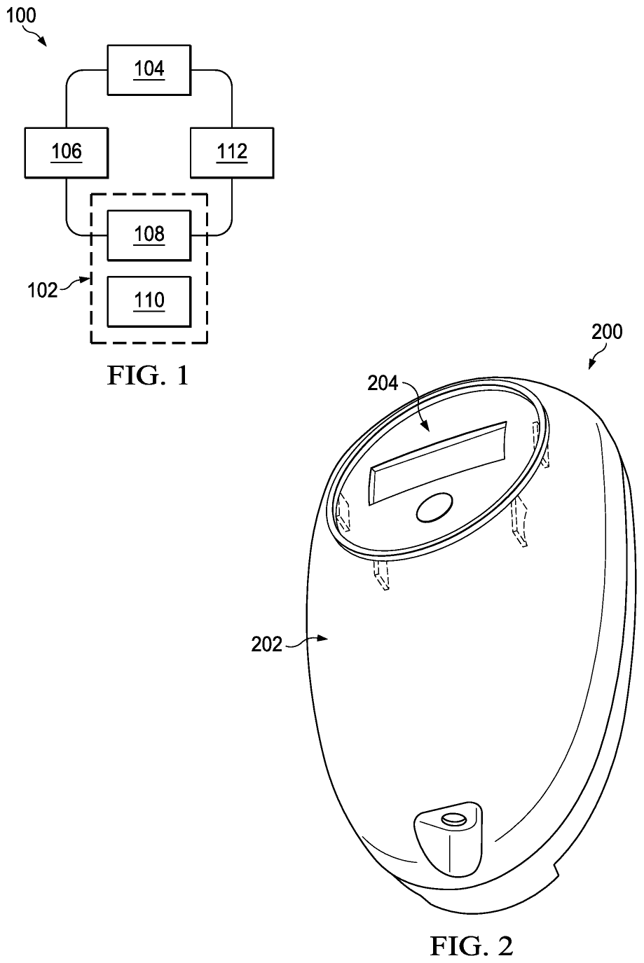

FIG. 1 illustrates an example embodiment of a therapy system 100 in accordance with this specification. The therapy system 100 may include a dressing and pressure source, such as a negative-pressure source. For example, a dressing 102 may be fluidly coupled to a pressure source 104, which may be a negative-pressure source 104. A controller 106 may also be fluidly and/or electronically coupled to the dressing 102 and the negative-pressure source 104. A dressing generally includes a cover and a tissue interface. The dressing 102, for example, may include a cover 108 and a tissue interface 110. The therapy system 100 may also include a fluid container, such as a container 112, coupled to the dressing 102 and to the negative-pressure source 104.

Components of the therapy system 100 may be coupled directly or indirectly. For example, the negative-pressure source 104 may be directly coupled to the controller 106 and indirectly coupled to the dressing 102 through the controller 106. In some embodiments, components may be coupled by virtue of physical proximity, being integral to a single structure, or being formed from the same piece of material. Coupling may also include mechanical, thermal, electrical, or chemical coupling or bonding in some contexts.

Components may also be fluidly coupled to each other to provide a path for transferring fluids, such as liquid and/or gas, between the components. In some embodiments, for example, components may be fluidly coupled through a fluid conductor. A “fluid conductor,” as used herein, broadly refers to a tube, pipe, hose, conduit, or other structure with one or more lumina adapted to convey a fluid between two ends. A tube, for example, is typically an elongated, cylindrical structure with some flexibility, but the geometry and rigidity may vary.

In operation, the tissue interface 110 may be placed within, over, on, or otherwise proximate to a tissue site. The cover 108 may be placed over the tissue interface 110 and sealed to tissue near the tissue site. For example, the cover 108 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 110 in the sealed therapeutic environment can induce macrostrain and microstrain in the tissue site, as well as remove exudates and other fluids from the tissue site, which can be collected in container 112 and disposed of properly.

The process of reducing pressure may be described illustratively herein as “delivering,” “distributing,” or “generating” negative pressure, for example. In general, exudates and other fluids flow toward lower pressure along a fluid path. Thus, in the context of a system for negative-pressure therapy, the term “downstream” may refer to a location in a fluid path relatively closer to a negative-pressure source, and conversely, the term “upstream” may refer to a location in a fluid path relatively further away from a negative-pressure source. Similarly, features may be described in terms of a fluid “inlet” or “outlet” in such a frame of reference. This orientation is generally presumed for purposes of describing various features and components of negative-pressure therapy systems herein. However, the fluid path may also be reversed in some applications (such as by substituting a positive-pressure source for a negative-pressure source), and thus, this descriptive convention should not be construed as limiting.

The term “tissue site” in this context broadly refers to a wound or defect located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be used in certain tissue areas to grow additional tissue that may be harvested and transplanted to another tissue location.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. In some examples, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure.

A pressure source, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure source may be housed within or used in conjunction with other components, such as sensors, processing units, alarm indicators, memory, databases, software, display devices, or user interfaces that further facilitate negative-pressure therapy. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

The tissue interface 110 can be adapted to contact a tissue site. The tissue interface 110 may be partially or fully in contact with the tissue site. If the tissue site is a wound, for example, the tissue interface 110 may partially or completely fill the wound, or may be placed over the wound. The tissue interface 110 may take many forms, and may have many sizes, shapes, or thicknesses depending on a variety of factors, such as the type of treatment being implemented or the nature and size of a tissue site. For example, the size and shape of the tissue interface 110 may be adapted to the contours of deep and irregular shaped tissue sites.

In some embodiments, the tissue interface 110 may be a manifold. A “manifold” in this context generally includes any substance or structure providing a plurality of pathways adapted to collect or distribute fluid across a tissue site under negative pressure. For example, a manifold may be adapted to receive negative pressure from a source and distribute the negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be channels interconnected to improve distribution or collection of fluids across a tissue site. For example, cellular foam, open-cell foam, reticulated foam, porous tissue collections, and other porous material such as gauze or felted mat generally include pores, edges, and/or walls adapted to form interconnected fluid pathways. Liquids, gels, and other foams may also include or be cured to include apertures and flow channels. In some illustrative embodiments, a manifold may be a porous foam material having interconnected cells or pores adapted to uniformly or quasi-uniformly distribute negative pressure to a tissue site. The foam material may be either hydrophobic or hydrophilic. In one non-limiting example, a manifold may be an open-cell, reticulated polyurethane foam such as GRANUFOAM® dressing available from Kinetic Concepts, Inc. of San Antonio, Tex.

In an example in which the tissue interface 110 may be made from a hydrophilic material, the tissue interface 110 may also wick fluid away from a tissue site, while continuing to distribute negative pressure to the tissue site. The wicking properties of the tissue interface 110 may draw fluid away from a tissue site by capillary flow or other wicking mechanisms. An example of a hydrophilic foam is a polyvinyl alcohol, open-cell foam such as V.A.C. WHITEFOAM® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 110 may further promote granulation at a tissue site when pressure within the sealed therapeutic environment is reduced. For example, any or all of the surfaces of the tissue interface 110 may have an uneven, coarse, or jagged profile that can induce microstrains and stresses at a tissue site if negative pressure is applied through the tissue interface 110.

In some embodiments, the tissue interface 110 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 110 may further serve as a scaffold for new cell-growth, or a scaffold material may be used in conjunction with the tissue interface 110 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 108 may provide a bacterial barrier and protection from physical trauma. The cover 108 may also be constructed from a material that can reduce evaporative losses and provide a fluid barrier between two components or two environments, such as between a therapeutic environment and a local external environment. The cover 108 may be, for example, an elastomeric film or membrane that can be sealed around a tissue site to maintain a negative pressure at the tissue site for a given negative-pressure source. In some example embodiments, the cover 108 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 108 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 108 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The container 112 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

FIG. 2 is a perspective view of a therapy unit 200, illustrating additional details that may be associated with some example embodiments of the therapy system 100. The therapy unit 200 may include an enclosure 202 for the pressure source 104 or negative pressure source, and may also include a user interface 204. In some embodiments, the therapy unit 200 may also integrate other components, such as the controller 106 or the container 112, for example.

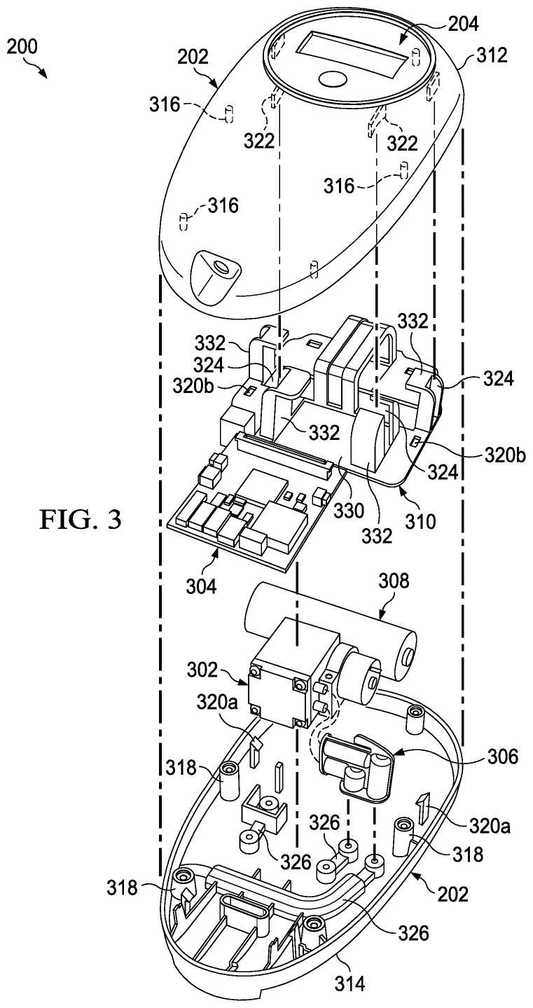

FIG. 3 is an exploded view of the therapy unit 200, illustrating additional details that may be associated with some embodiments. As illustrated in the example embodiment of FIG. 3, the therapy unit 200 may include the enclosure 202, a pressure source 302, a control board 304, a pneumatic coupler 306, a power source 308, and a multi-function mount 310. The pressure source 302 is an example embodiment of the negative-pressure source 104 in FIG. 1. In some example embodiments, the power source 308 may be, without limitation, a battery, capacitor, transformer, regulator, or electrical adapter configured to receive and to convert power from an outside source, such as a wall outlet, for use with the therapy unit 200.

The control board 304 is an example embodiment of the controller 106 in FIG. 1. The control board 304 may be configured to control the pressure source 302 and may be positioned in the enclosure 202 in communication with the power source 308 and the pressure source 302. The control board 304 may control the pressure source 302, for example, according to a logic control algorithm stored on or associated with the control board 304, a user input from the user interface 204, or a signal from a sensor. The control board 304 may include or communicate with various sensors, such as a pressure transducer, and one or more valves, such as a solenoid valve, to suit a particular application.

Components of the therapy system 100 and the therapy unit 200 may be omitted or additional components may be added in various embodiments to suit a particular application. Accordingly, components or features described in the example embodiments herein may not be deemed essential or required to practice the invention as defined by the appended claims.

As illustrated in FIG. 3, some embodiments of the enclosure 202 may include a first housing 312 and a second housing 314, which may be coupled to form the enclosure 202 and to enclose components of the therapy unit 200. The first housing 312 may include the user interface 204 in some embodiments. Further, the first housing 312 may include a plurality of first mating stand-offs 316 and the second housing 314 may include a plurality of second mating stand-offs 318. The first mating stand-offs 316 may be configured to mate with the second mating stand-offs 318 for coupling the first housing 312 to the second housing 314. A gasket (not shown) or an elastomeric interface button may be located, for example, between the first housing 312 and the second housing 314 to provide additional sealing or further functionality.

In some embodiments, the multi-function mount 310 may be coupled to the enclosure 202 by a snap-fit assembly 320, such as, for example, a protrusion 320a and a catch 320b configured to mate with one another. The protrusion 320a or the catch 320b may be configured to yield or deflect when inserted into or brought into contact with one another such that mating surfaces of the protrusion 320a and the catch 320b become coupled. Further, in some embodiments, the multi-function mount 310 may be supported within the enclosure 202 by a plurality of support tabs 322. The support tabs 322 may be configured to contact the multi-function mount 310 within the enclosure 202.

In some embodiments, the second housing 314 may be coupled to the multi-function mount 310, and the first housing 312 may include the plurality of support tabs 322. The support tabs 322 may extend outward from the first housing 312 and may be configured contact and to support the multi-function mount 310 when the first housing 312 is coupled to the second housing 314. In some embodiments, the support tabs 322 may exert a compressive force on the multi-function mount 310 when the first housing 312 is coupled to the second housing 314. Further, in some embodiments, the support tabs 322 may be received within or aligned relative to a pocket 324 or surface feature of the multi-function mount 310 to provide support or to prevent lateral movement of the multi-function mount 310 within the enclosure 202.

Referring to FIGS. 3-5, in some embodiments, the enclosure 202 may include at least one pneumatic conduit 326. The pneumatic conduit 326 may be, for example, a fluid conductor, a fluid pathway, or a conduit integrally formed as part of the enclosure 202, molded within a substrate material of the enclosure 202, or coupled to a surface of the enclosure 202. In embodiments in which the pneumatic conduit 326 is formed from or as part of the substrate material of the enclosure 202, the pneumatic conduit 326 may be referred to as an integrated pneumatic conduit. As a further example, the pneumatic conduit 326 may be formed as a channel, groove, furrow, cut, depression, or gutter in or within a surface of the enclosure 202. In some embodiments, the enclosure 202 may be manufactured, in whole or in part, with a molding process, such as injection molding.

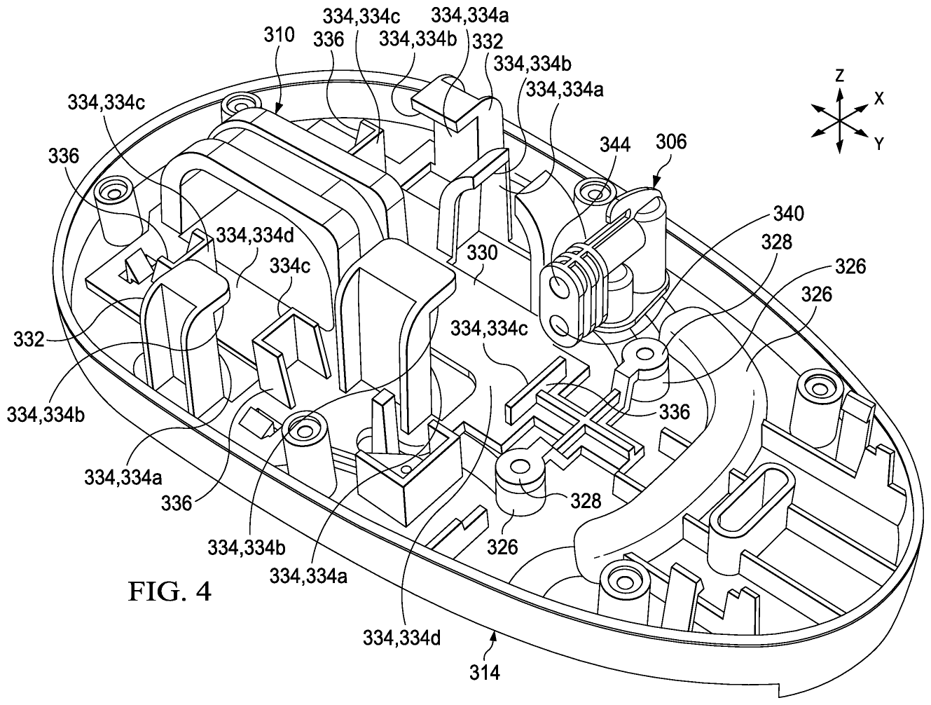

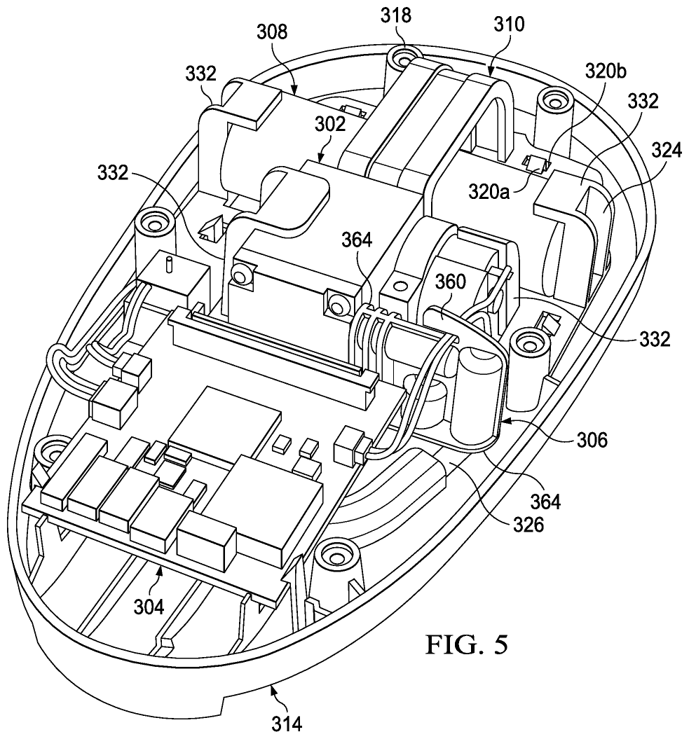

Referring to FIGS. 4-5, the multi-function mount 310 may be coupled within the enclosure 202. In some embodiments, the pressure source 302 and the power source 308 may be carried by the multi-function mount 310. In some embodiments, the multi-function mount 310 may include or be formed of a resilient material configured to dampen vibration. For example, the multi-function mount 310 may include a semi-rigid material having a hardness between 20 Shore A to 75 Shore A. In some embodiments and without limitation, the multi-function mount 310 may be manufactured from a thermoplastic elastomer material, such as, for example, KRAIBURG THERMOLAST K TF5 STE.

In some embodiments, the multi-function mount 310 may include one or more pneumatic seals 328, which may be integrally formed as part of the multi-function mount 310 and configured to provide a pneumatic seal relative to the at least one pneumatic conduit 326 within the enclosure 202. The control board 304 and other components of the therapy unit 200 may include one or more suitable pneumatic connections configured to be coupled to or sealingly engaged with the pneumatic seal 328 carried by the multi-function mount 310. As shown in the example of FIG. 4, the pneumatic seals 328 may have a circular or annular shape configured to seal around a port, aperture, or opening in fluid communication with one or more of the pneumatic conduits 326. The pneumatic seals 328 may have other shapes or configurations in other embodiments. Further, the positioning or integration of the pneumatic seals 328 as a component of the multi-function mount 310 may enhance assembly by automatically locating and positioning the pneumatic seals 328 relative to the pneumatic conduits 326 when the multi-function mount 310 is coupled to the enclosure 202.

In some embodiments, the multi-function mount 310 may include a mount base 330 and one or more capturing members 332 configured to extend outward from the mount base 330. At least one of the capturing members 332 may be configured to contact the power source 308 or the pressure source 302 on at least two contact surfaces 334, which may be non-coplanar contact surfaces. For example, at least one of the capturing members 332 may be configured to contact the power source 308 or the pressure source 302 at a first contact surface 334a and a second contact surface 334b positioned non-coplanar to the first contact surface 334a. The capturing members 332 may be configured to provide an interference fit with the pressure source 302 or the power source 308.

In some embodiments, the multi-function mount 310 may include one or more isolation bosses 336. The one or more isolation bosses 336 may be configured to contact the power source 308 or the pressure source 302 at a third contact surface 334c positioned non-coplanar to the first contact surface 334a and the second contact surface 334b.

In some embodiments, the mount base 330 may be configured to contact the pressure source 302 or the power source 308 at a fourth contact surface 334d positioned non-coplanar to the first contact surface 334a, the second contact surface 334b, and the third contact surface 334c. The first contact surface 334a, the second contact surface 334b, the third contact surface 334c, and the fourth contact surface 334d may each lie in a separate plane positioned normal to or facing one another.

As used in this disclosure, the term non-coplanar may refer to a point or surface that does not lie in the same geometric plane as another point or surface. In some embodiments, the first contact surface 334a of one or more of the capturing members 332 may lie in a separate plane opposite from and facing the first contact surface 334a of another of the capturing members 332. In this configuration, the first contact surface 334a of one of the capturing members 332 may face the first contact surface 334a of another of the capturing members 332 such that the pressure source 302 or the power source 308 may be captured between the opposing first contact surfaces 334a, preventing translational or other movement of the pressure source 302 or the power source 308 along an x-axis. Further, in some embodiments, the third contact surface 334c of one or more of the isolation bosses 336 may lie in a separate plane opposite from and facing the third contact surface 334c of another of the isolation bosses 336. In this configuration, the third contact surface 334c of one of the isolation bosses 336 may face the third contact surface 334c of another of the isolation bosses 336 such that the pressure source 302 or the power source 308 may be captured between the opposing third contact surfaces 334c, preventing translational or other movement of the pressure source 302 or the power source 308 along a y-axis. Further, in some embodiments, the second contact surface 334b of one or more of the capturing members 332 may lie in a separate plane and opposite from and facing the fourth contact surface 334d of the mount base 330. In this configuration, the second contact surface 334b may face the fourth contact surface 334d such that the pressure source 302 or the power source 308 is captured between the opposing second contact surface 334b and the fourth contact surface 334d, preventing translational or other movement of the pressure source 302 or the power source 308 along a z-axis. The x-axis, y-axis, and z-axis described in the above embodiments may be positioned normal or perpendicular relative to one another as shown in FIG. 4. Preventing translational or other movement of the pressure source 302 or the power source 308 along one or more of the axes as described in these example embodiments may provide support and additionally prevent vibration from the pressure source 302 or the power source 308 from being communicated to other parts of the enclosure 202.

Referring to FIGS. 3-6, the pneumatic coupler 306 may be configured to fluidly couple the pressure source 302 within the enclosure 202 to the therapy unit 200 and other components of the therapy system 100. The pneumatic coupler 306 may be a separate component coupled to the therapy unit 200 or the multi-function mount 310, for example. In other example embodiments, the pneumatic coupler 306 may be formed integrally with the therapy unit 200 or the multi-function mount 310. Further, in some embodiments, the pneumatic coupler 306 may be configured to support the pressure source 302 within the enclosure 202 and to prevent vibrations from being transmitted to the enclosure 202. For example, at least by virtue of being a single integrated and separable component having separate connectivity within the enclosure 202, in some embodiments, the pneumatic coupler 306 may provide improved reduction in both pneumatic noise and mechanical noise, which may be associated with or created by the operation of the pressure source 302. Further, the pneumatic coupler 306 may provide an additional mounting point or connection point between the pressure source 302 and the enclosure 202, which may provide additional support and stability to the pressure source 302 within the enclosure 202.

Referring to FIGS. 4-7, the pneumatic coupler 306 may include a source inlet port 340 in fluid communication with a mount inlet port 342 through the pneumatic coupler 306 and a source outlet port 344 in fluid communication with a mount outlet port 346 through the pneumatic coupler 306.

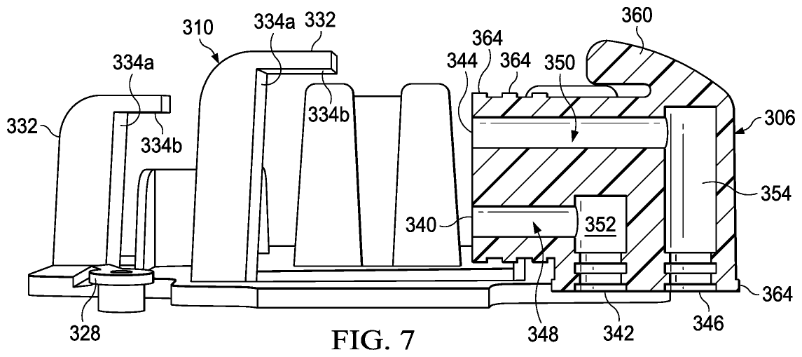

Referring to FIG. 7, the source inlet port 340 is in fluid communication with the mount inlet port 342 through an inlet pathway 348 disposed within the pneumatic coupler 306. The source outlet port 344 is in fluid communication with the mount outlet port 346 through an outlet pathway 350 disposed within the pneumatic coupler 306. The inlet pathway 348 and the outlet pathway 350 are separate from one another. In some embodiments, the pneumatic coupler 306 may include an inlet expansion chamber 352 within the inlet pathway 348 and an outlet expansion chamber 354 within the outlet pathway 350 disposed through the pneumatic coupler 306. The inlet expansion chamber 352 may increase a volume of a portion of the inlet pathway 348, and the outlet expansion chamber 354 may increase a volume of a portion of the outlet pathway 350. The volume, diameter, or size of the inlet expansion chamber 352 or the outlet expansion chamber 354 may be adjusted in some embodiments to reduce sound levels. The volume of the inlet expansion chamber 352 and the outlet expansion chamber 354 may be sized sufficiently to dissipate fluid flow acoustics or pressure spikes that may be created by the pressure source 302. In some embodiments, the inlet expansion chamber 352 or the outlet expansion chamber 354 may also include baffles or sound-absorbing foam to further reduce sound associated with fluid flow from the pressure source 302.

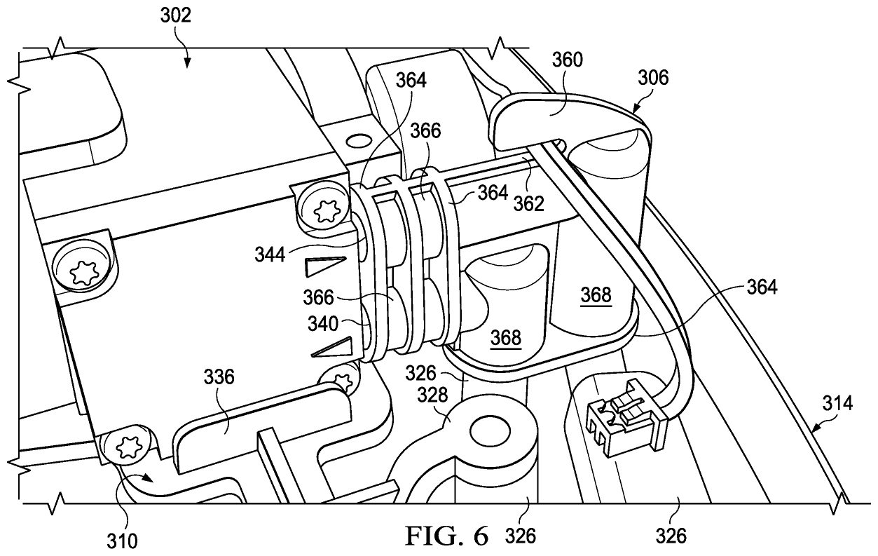

Referring to FIG. 6, in some embodiments, the pneumatic coupler 306 may include a cable guide 360. In some embodiments, the cable guide 360 may extend outward from and over-lapping an exterior surface 362 of the pneumatic coupler 306 to define a three-sided surrounding relative to the exterior surface 362 of the pneumatic coupler 306 that is configured to support a cable or wire therein. In other embodiments, the cable guide 360 may be configured as a tab including a hole or aperture sized to receive a cable or wire and associated connectors. The cable guide 360 may simplify assembly and reduce stress on components of the therapy unit 200, which may be caused by routing cables or wires to the components.

Further, in some embodiments, the pneumatic coupler 306 may include at least one reinforcement rib 364 extending outward from and surrounding a perimeter 366 of the source inlet port 340 and the source outlet port 344. In some embodiments, the at least one reinforcement rib 364 may additionally or alternatively be configured or positioned to extend outward from and surrounding a perimeter 368 of the mount inlet port 342 and the mount outlet port 346. The at least one reinforcement rib 364 may add stiffness and rigidity to help reduce or eliminate material creep or movement in the pneumatic coupler 306, which may improve the sealing ability and reliability of the pneumatic coupler 306, particularly after extended periods of storage or use.

In operation, the therapy unit 200 may be coupled to a canister, such as the container 112 of FIG. 1, which can be fluidly coupled to a dressing, such as the dressing 102 of FIG. 1. The pressure source 302 can produce a prescribed negative pressure, which can be distributed to the container 112 through a fluidic connection. The negative pressure can then be distributed through the container 112 to the dressing 102. In some embodiments, the container 112 may be omitted or positioned within the enclosure 202 of the therapy unit 200 in which a direct fluidic connection may be made between the therapy unit 200 and the dressing 102.

Many negative-pressure therapy systems may use a reciprocating diaphragm or piston pump to generate negative pressure for therapy. For example, the pressure source 302 may be a reciprocating pump. In operation, this type of pump typically emits pulses or pressure waves, which can create noise and vibration. Noise can be particularly problematic in smaller pumps that produce relatively high airflow rates, since smaller pumps generally rotate at a higher speed to produce higher flow rates. Excessive noise can interfere with patient compliance, particularly in public places or at night.

In some embodiments, a plenum, extended fluid pathway, or expansion chamber, such as illustrated by the inlet expansion chamber 352 and the outlet expansion chamber 354 associated with the pneumatic coupler 306 can reduce pressure peaks of air flow, reducing the sound level of an apparatus without significantly increasing the size or cost of an apparatus. Baffles, sound-absorbing foam, or both may additionally or alternatively be used to reduce the sound level.

For example, positive pressure fluid flow from an outlet port of the pressure source 302 may enter the outlet expansion chamber 354 of the pneumatic coupler 306 through the source outlet port 344. Within the outlet expansion chamber 354, sound waves may be reflected and interfere with each other, creating a noise cancelling effect for reducing sound levels before leaving the outlet expansion chamber 354 through the mount outlet port 346 of the pneumatic coupler 306. In an analogous manner, negative pressure fluid flow may be drawn into the inlet expansion chamber 352 from the mount inlet port 342 of the pneumatic coupler 306 and toward an inlet port of the pressure source 302 fluidly coupled to the source inlet port 340 of the pneumatic coupler 306. Noise cancellation and sound reduction may similarly occur within the inlet expansion chamber 352 as previously described for the outlet expansion chamber 354. As described herein, the multi-function mount 310 and the pneumatic coupler 306 may provide a beneficial reduction in operational noise, simplified pneumatic connections, and simplified assembly for the therapy unit 200.

While shown in a few illustrative embodiments, a person having ordinary skill in the art will recognize that the systems, apparatuses, and methods described herein are susceptible to various changes and modifications. Features may be emphasized in some example embodiments while being omitted in others, but a person of skill in the art will appreciate that features described in the context of one example embodiment may be readily applicable to other example embodiments. Moreover, descriptions of various alternatives using terms such as “or” do not require mutual exclusivity unless clearly required by the context, and the indefinite articles “a” or “an” do not limit the subject to a single instance unless clearly required by the context.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A therapy unit for treating a tissue site, comprising:
an enclosure;
a multi-function mount coupled within the enclosure;
a power source carried by the multi-function mount;
a pressure source carried by the multi-function mount; and
a pneumatic coupler configured to fluidly couple the pressure source to the therapy unit and comprising an inlet expansion chamber within an inlet pathway and an outlet expansion chamber within an outlet pathway disposed through the pneumatic coupler.

2. (canceled)

3. The therapy unit of claim 1, wherein the enclosure comprises a first housing and a second housing, wherein the second housing is coupled to the multi-function mount, and wherein the first housing comprises a plurality of support tabs extending outward from the first housing and configured contact and to support the multi-function mount when the first housing is coupled to the second housing.

4. The therapy unit of claim 3, wherein the first housing comprises a plurality of first mating stand-offs and the second housing comprises a plurality of second mating stand-offs, and wherein the first mating stand-offs are configured mate with the second mating stand-offs for coupling the first housing to the second housing.

5. The therapy unit of claim 3, wherein the plurality of support tabs exert a compressive force on the multi-function mount when the first housing is coupled to the second housing.

6. The therapy unit of claim 1, wherein the enclosure comprises at least one integrated pneumatic conduit.

7. (canceled)

8. (canceled)

9. The therapy unit of claim 1, wherein the multi-function mount comprises at least one pneumatic seal integrally formed as part of the multi-function mount and configured to provide a pneumatic seal relative to at least one pneumatic conduit within the enclosure.

10. The therapy unit of claim 1, wherein the multi-function mount comprises one or more capturing members, wherein at least one of the capturing members is configured to contact the power source or the pressure source on at least two non-coplanar surfaces.

11. (canceled)

12. The therapy unit of claim 1, wherein the multi-function mount comprises one or more capturing members, wherein at least one of the capturing members is configured to contact the power source or the pressure source at a first contact surface and a second contact surface positioned non-coplanar to the first contact surface.

13. The therapy unit of claim 12, wherein the multi-function mount further comprises one or more isolation bosses configured to contact the power source or the pressure source at a third contact surface positioned non-coplanar to the first contact surface and the second contact surface.

14. The therapy unit of claim 13, wherein the multi-function mount further comprises a mount base configured to contact the power source or the pressure source at a fourth contact surface positioned non-coplanar to the first contact surface, the second contact surface, and the third contact surface.

15. The therapy unit of claim 14, wherein the first contact surface, the second contact surface, the third contact surface, and the fourth contact surface each lie in a separate plane positioned normal to one another.

16. (canceled)

17. The therapy unit of claim 1, wherein the pneumatic coupler further comprises a source inlet port in fluid communication with a mount inlet port through the pneumatic coupler and a source outlet port in fluid communication with a mount outlet port through the pneumatic coupler.

18. The therapy unit of claim 17, wherein the source inlet port is in fluid communication with the mount inlet port through the inlet pathway disposed within the pneumatic coupler, and wherein the source outlet port is in fluid communication with the mount outlet port through the outlet pathway disposed within the pneumatic coupler, and wherein the inlet pathway and the outlet pathway are separate from one another.

19. The therapy unit of claim 17, wherein the pneumatic coupler further comprises at least one reinforcement rib extending outward from and surrounding a perimeter of the source inlet port and the source outlet port.

20. The therapy unit of claim 17, wherein the pneumatic coupler further comprises at least one reinforcement rib extending outward from and surrounding a perimeter of the mount inlet port and the mount outlet port.

21. The therapy unit of claim 1, wherein the inlet expansion chamber increases a volume of a portion of the inlet pathway, and wherein the outlet expansion chamber increases a volume of a portion of the outlet pathway.

22. The therapy unit of claim 1, wherein the pneumatic coupler further comprises a cable guide extending outward from and over-lapping an exterior surface of the pneumatic coupler to define a three-sided surrounding relative to the exterior surface of the pneumatic coupler.

23. The therapy unit of claim 1, wherein the pneumatic coupler is configured to support the pressure source within the enclosure and to prevent vibrations from being transmitted to the enclosure.

24. (canceled)

25. (canceled)

26. The therapy unit of claim 1, further comprising a control board positioned in the enclosure in communication with the power source and the pressure source, wherein the control board is configured to control the pressure source according to a user input or a sensor signal.

27. The therapy unit of claim 26, wherein the control board comprises at least one pneumatic connection configured to be coupled to at least one pneumatic seal carried by the multi-function mount.

28.-47. (canceled)
